(19) United States
(12) Patent Application Publication
(10) Pub. No.: US 2024/0099729 A1
(43) Pub. Date: Mar. 28, 2024

(54) BONE MARROW ASPIRATE HARVESTING

(71) Applicant: Biogennix, LLC, Irvine, CA (US)

(72) Inventor: Mark Borden, Boulder, CO (US)

(21) Appl. No.: 18/373,161

(22) Filed: Sep. 26, 2023

Related U.S. Application Data
(60) Provisional application No. 63/409,883, filed on Sep. 26, 2022.

(51) Int. Cl. A61B 17/16; A61B 17/34

(52) U.S. Cl. CPC A61B 17/1635; A61B 17/3472; A61B 17/3476; A61B 2017/00561

(57) ABSTRACT

Bone marrow aspirate harvesting is provided via use of a surgical device that includes a needle including a plurality of lateral holes defined at different positions longitudinally along a wall thereof and a movable aspiration insert within the needle, wherein the needle need not be repositioned within the patient to harvest bone marrow aspirate. The surgical device includes a needle with lateral holes; a movable aspiration insert including an aspiration window that is sized to interface with one lateral hole of the plurality of lateral holes at a time, wherein the aspiration insert is disposed in a lumen of the needle; and a handle including a positioning means in communication with the aspiration insert, wherein the positing means is configured to actuate between a first state and a second state to move the aspiration insert a certain distance in a first direction along a longitudinal axis of the needle.

### CROSS REFERENCES TO RELATED APPLICATIONS

The present disclosure claims the benefit of U.S. Provisional Patent Application No. 63/409,883 entitled “BONE MARROW ASPIRATE HARVESTING” filed on 2022 Sep. 26, which is incorporated herein by reference in its entirety

### BACKGROUND

Bone marrow is present in cancellous bones and the medullary canal found throughout the skeleton. Bone marrow is a semi-solid tissue that consists of various cells, adipose tissue (fat), and blood vessels. Within the cellular component, one specialized group of cells, mesenchymal stem cells, represents a highly useful cell type that can differentiate into a variety of tissue specific cells (osteoblasts, chondrocytes, myocytes, etc.). As such, bone marrow aspirate is often used in various surgical and medical procedures to augment the healing process. For example, surgeons frequently combine BMA with bone graft materials to create a biological combination of stem cells (capable of turning into osteoblasts) and a tissue regeneration scaffold (bone graft) that can improve the bone formation response.

### SUMMARY

The present disclosure is generally related to an improved Bone Marrow Aspirate (BMA) harvesting device and improved methods of BMA harvesting using such a device.

For the various surgical/medical applications of bone marrow, obtaining BMA with a high stem cell concentration is an important consideration, since higher stem cell counts provide a greater biological benefit. Conversely, if the stem cell concentration is too low, then the healing benefits are reduced or fully lost. Accordingly, to collect sufficient quantiles of BMA, in a manner that is minimally invasive to the patient, various surgical tools are used and medical personnel are trained in specific techniques that ensure BMA is harvested with a high concentration of stem cells.

The described devices and methods provide for improvements over the conventional tools and techniques, thereby leading to a higher quality of the BMA collected from a given site, and improved ease of use, thereby reducing the likelihood of operator error, reducing operator fatigue, decreasing the amount of time to collect sufficient BMA from a patient, and increasing speed of training.

One embodiment of the present disclosure is a surgical device, comprising: a needle including a plurality of lateral holes defined at different positions longitudinally along a wall of the needle, wherein each lateral hole of the plurality of lateral holes is located a certain distance from at least one adjacent lateral hole of the plurality of lateral holes; an aspiration insert including an aspiration window that is sized to interface with one set of lateral holes of the plurality of lateral holes at a time, wherein the aspiration insert is disposed in a lumen of the needle; and a handle including a trigger in communication with the aspiration insert, wherein the trigger is configured to actuate between a first state and a second state to move the aspiration insert a certain distance in a first direction along a longitudinal axis of the needle.

One embodiment of the present disclosure is a method, comprising: applying a negative pressure to a first bone marrow collection site via an aspiration insert having an aspiration window aligned with a first set of lateral holes defined in a needle in which the aspiration insert is disposed; actuating a trigger to retract the aspiration insert by a certain distance relative to the needle while the needle remains stationary relative to the first bone marrow collection site to align the aspiration window with a second lateral hole defined in the needle; and applying the negative pressure to a second bone marrow collection site via the aspiration insert when aligned with the second lateral hole defined in the needle.

One embodiment of the present disclosure is a surgical device, comprising: a needle including a first lumen and a plurality of sets of lateral holes defined at a plurality of positions in a wall of the needle, wherein each lateral hole of a given set of lateral holes defined at a given position of the plurality of positions is defined circumferentially around the wall, and wherein each position of the plurality of positions is located a certain distance longitudinally along the needle from at least one adjacent position of the plurality of positions; an aspiration insert, disposed in the first lumen, and including a second lumen and an aspiration window; and a handle including a positioning means configured to, upon actuation, reposition the aspiration window from alignment with a first set of lateral holes of the plurality of sets of lateral holes defined at a first position of the plurality of positions to a second set of lateral holes of the plurality of sets of lateral holes to place the second lumen in fluid communication with a different portion of an environment outside of the first lumen.

### BRIEF DESCRIPTION OF THE DRAWINGS

The accompanying figures depict various elements of the one or more embodiments of the present disclosure, and are not considered limiting of the scope of the present disclosure.

In the Figures, some elements may be shown not to scale with other elements so as to more clearly show the details. Additionally, like reference numbers are used, where possible, to indicate like elements throughout the several Figures. Additionally, although primarily illustrated with a right-handled configuration, the various features may be mirrored across an axis to provide for a left-handed configuration.

It is contemplated that elements and features of one embodiment may be beneficially incorporated in the other embodiments without further recitation or illustration. For example, as the Figures may show alternative views and time periods, various elements shown in a first Figure may be omitted from the illustration shown in a second Figure without disclaiming the inclusion of those elements in the embodiments illustrated or discussed in relation to the second Figure.

FIGS. 1A and 1B illustrate an example harvesting device, according to embodiments of the present disclosure.

FIGS. 2A-2C illustrate detailed views of the needles of a harvesting device with associated movable aspiration inserts, according to embodiments of the present disclosure.

FIGS. 3A and 3B illustrate cross-sectional views of the needles of a harvesting device with varying lateral hole patterns taken in different planes perpendicular to the longitudinal axis of the needles, according to embodiments of the present disclosure.

FIG. 4 illustrates an impact plate for insertion of a harvesting device to an extraction site, according to embodiments of the present disclosure.

FIG. 5 is a flowchart of a method for Bone Marrow Aspirate harvesting using a described surgical device, according to embodiments of the present disclosure.

FIGS. 6A and 6B illustrate the alignment groove within the aspiration insert which maintains rotational position and ensures the aspiration window aligns with the lateral holes on the needle.

### DETAILED DESCRIPTION

The present disclosure is generally related to an improved Bone Marrow Aspirate (BMA) harvesting device and improved methods of BMA harvesting using such a device. The described device allows users to collect a greater amount of BMA from a given site, with a higher concentrations of desired stem cells, compared to other devices. Additionally, the design provides for a two-handed mode of use, rather than the “three-handed” mode of use found in other devices where the device must be controlled or actuated at three or more separate locations; thereby allowing continuous use by a single operator versus two operators or continuous operation by a single operator versus discontinuous operation by a single operator.

One challenge with harvesting bone marrow is the difficulty of obtaining sufficient material while maintaining a high stem cell concentration, due (in part) to the propensity of the bone marrow harvesting technique to dilute the aspirate with peripheral blood. While trephine-based harvesting can remove an intact bone marrow biopsy with little dilution from peripheral blood, this technique produces small volumes of tissue that are not sufficiently sized for most surgical and medical procedures. Additionally, the trephine-based technique also removes a core of cancellous bone that is not desirable for non-bone related applications, such vertebral disc regeneration or cartilage repair. As a result, the large majority of bone marrow harvesting is conducted using needle-based systems that suction bone marrow out of the harvest site. This type of harvesting creates a fluid referred to as bone marrow aspirate (BMA), which due to its fluid form, can be easily injected to other sites within the body or be combined with tissue regeneration materials, such as bone grafts. Typical BMA harvest sites are found in cancellous bones that have a high concentration and volume of bone marrow and are easily accessible through Minimally Invasive Surgery (MIS) or percutaneous harvesting techniques, such as the iliac crest, vertebral bodies, the distal and proximal tibia, and the calcaneus.

Accordingly, standard operating procedure for the aspiration technique indicates that no more than 1-2 cubic centimeters (cc) of BMA should be aspirated from any given needle location to avoid suctioning surrounding peripheral blood and lowering the stem cell concentration of the BMA. Because bone marrow is more viscous compared to blood, operators need to reposition the collecting device once 1-2 cc of BMA is collected from a given site, otherwise continued aspiration in the same location pulls in blood rather than the surrounding bone marrow, which dilutes the BMA and reduces the stem cell concentrations. Although this technique sounds simple in theory, in practice, poor execution of the technique coupled with limitations of the prior harvesting devices can make proper aspiration difficult to perform, time consuming, and prone to errors that can reduce the quality of the BMA collected, which, quickly causes stem cell concentrations to drop and eliminates or reduces the biological benefit.

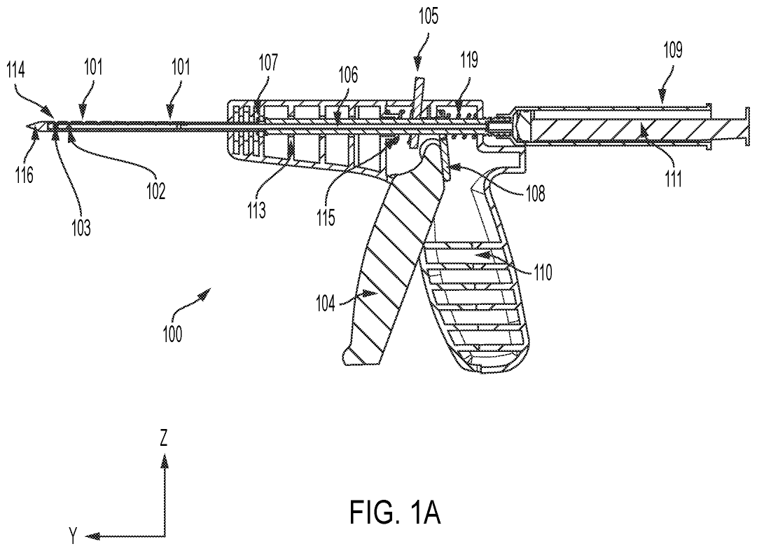

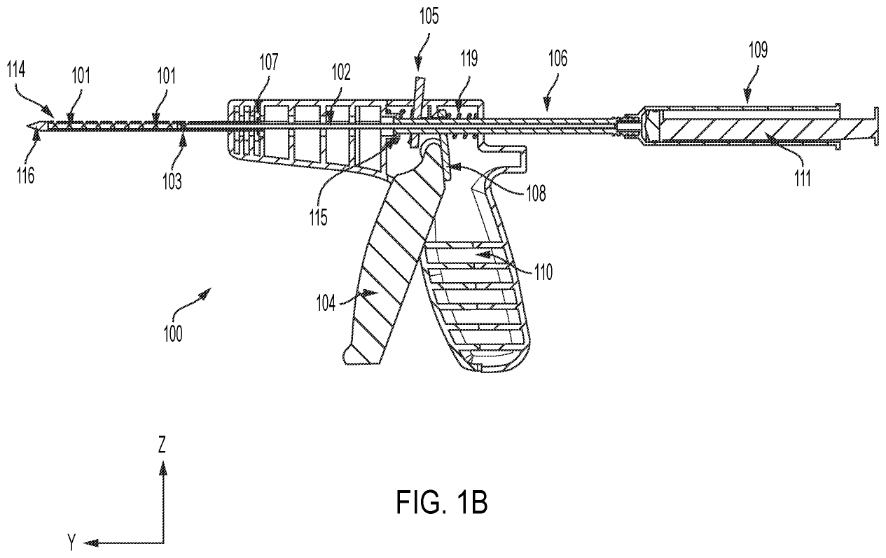

FIGS. 1A and 1B illustrate an example harvesting device 100, according to embodiments of the present disclosure. The harvesting device 100 is a surgical device for the collection of BMA, which improves upon and address the issues associated with standard needle aspiration devices. The improvement is accomplished by increasing the number of simultaneous aspiration areas, reducing device related harvesting steps, increasing ease-of-use, and minimizing technique errors. These improvements ensure that high stem cell count BMA is harvested and reduce the risk of technique errors that negatively impact BMA quality.

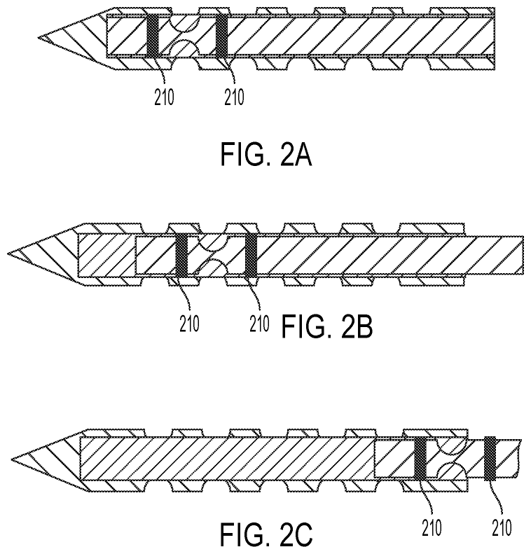

FIG. 1A illustrates the harvesting device 100 in an initial configuration, where a distal end of an aspiration insert 102 is fully inserted into the lumen of a needle 114, as the aspiration insert 102 may be positioned at the initiation of a BMA harvesting session. FIG. 2B illustrates the harvesting device 100 in a subsequent configuration, where a distal end of an aspiration insert 102 has been retracted from the initial position in the lumen of a needle 114, as the aspiration insert 102 may be repositioned during a BMA harvesting session while leaving the needle 114 stationary in the patient.

As illustrated, the harvesting device 100 uses a needle 114 that has a wall through which a plurality of lateral holes 101 are defined to access an internal lumen. A first end of the needle 114 ends with an integrated trocar tip 116 that fully blocks off the first end of the needle 114 and provides for easier insertion through cortical bone to a bone marrow collection site. The second end of the needle 114, opposite to the first end, is open, and may end with a needle collet 107 to help secure the needle within the handle 110 (as illustrated in FIGS. 1A and 1B). The needle collet 107 can be adhered to the handle 110 through the use of adhesives, ultrasonic welding, or other means. The second end of the needle 114 may be flared outward to fully embed the needle end in the needle collet 107. In another embodiment, the second end of the needle 114, opposite to the first end, is open, and may end with an abutment to an impact transfer plate or flare outward relative to a longitudinal axis of the needle 114 (e.g., for the needle 114 to provide an integrated transfer plate). In various embodiments, when the needle 114 is flared outward on the second end, the integrated impact transfer plate helps secure the needle 114 within the handle 110 of the harvesting device 100.

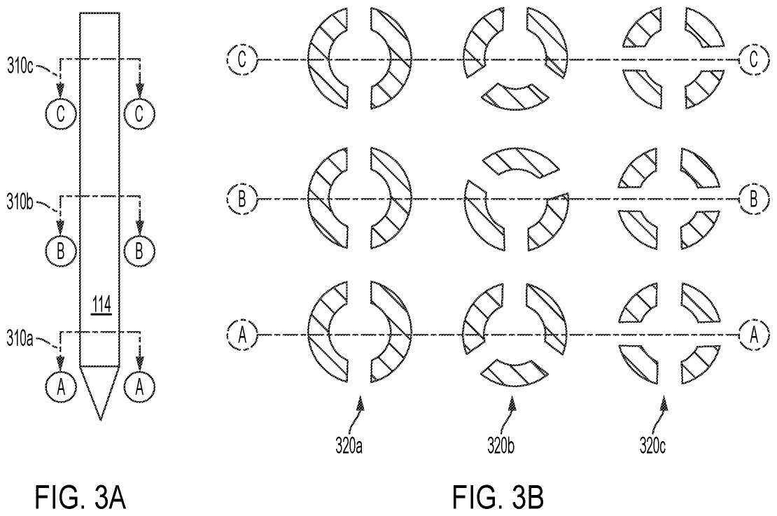

In various embodiments, the needle 114 is machined out of cylindrical stainless steel rod as a single piece or machined from thick walled tube stock, and has a wall thickness greater than prior art BMA harvesting needles fabricated according to the French scale. For example, the needle 114 may have walls with a thickness of at least 0.38 mm (0.015 in). The shape, number, and arrangements of the lateral holes 101 may vary in different embodiments, and are discussed in greater detail in regard to FIGS. 2A-3B. By using a thicker-walled needle 114, the presently described design is more robust against mechanical failure and may include a greater number (or greater size) of lateral holes 101 than thinner-walled designs. In various embodiments, the thicker-walled design may have a larger, smaller, or similarly sized outer diameter to thinner-walled designs based on the inner bore of the lumen defined within the needle 114. Accordingly, the needle 114 may have an outer diameter equal to a French scale needle, but an inner lumen that is smaller than the French scale needle of equivalent outer diameter, or may have an inner lumen having a bore equal to a French scale needle, but an outer diameter that is greater than the French scale needle of equivalent inner bore, while providing a thicker wall than the example French scale needles.

The aspiration insert 102 includes one or more aspiration windows 103 that are configured to mate with the lateral holes 101 defined in the needle 114 and to move within the internal lumen of the needle 114 to reposition the aspiration window(s) 103 with different lateral holes 101 to change the bone marrow harvest location, from which BMA is harvested. Due to this design, rather than repositioning the needle 114 in the patient's bone, as seen with standard BMA devices, an operator may leave the needle 114 stationary (relative to the bone marrow extraction sites) and move the aspiration insert 102 to define different active BMA harvesting locations. The elimination of repositioning the needle 114 to engage new harvesting locations minimizes any damage to the marrow cavity and disruption of capillaries that could cause excessive bleeding and allow BMA dilution to occur. In various embodiments, various positioning means can be used including threads, screws, gears, ratchet mechanisms, and the like.

In the example illustrated in FIGS. 1A and 1B, the positioning means includes a trigger 104, an advancer release 105, and an insert advancer 108 that are in communication with the aspiration insert collet 106. In various embodiments, the positioning means may additionally or alternatively include a ratchet mechanism, a gear mechanism, or a rotational knob that allows a user to precisely position the aspiration insert 102 within the needle 114. The various positioning means enable “two-handed” control of the device and provide various tactile and visual indications of operation and how the device is set.

When an operator actuates the trigger 104, the insert advancer 108 engages the aspiration insert collet 106 surrounding a portion of the aspiration insert 102 and pulls the aspiration insert 102 outward from the tip of the needle 114. The amount of distance that the insert advance 108 pulls the aspiration insert 102 is tuned to the distance to align the aspiration window 103 to the next lateral hole 101 in a series of lateral holes 101 defined along the length of the needle 114. Stated differently, with a single pull of the trigger 104, the insert advancer 108 moves the aspiration insert 102 from one hole 101 to a new hole 101 to access a new BMA harvesting location. The design may also include a mechanical feedback feature, such as a ball-detent, on the aspiration insert collet 106 that provides an audible or tactile signal when the aspiration insert 102 is properly positioned at the next set of lateral holes 101.

Similarly to the insert advancer 108, the advancer release 105 disengages from the aspiration insert collet 106 surrounding a portion of the aspiration insert 102 when actuated, and allows the aspiration insert 102 to freely slide within the needle 114 and be repositioned to the first closed end of the needle 114. This is done when the device 100 needs to be reset and placed in a new bone marrow site. Otherwise, when not actuated, the advancer release 105 holds the aspiration insert 102 in place relative to the needle 114, unless the trigger 104 is pulled.

One or more springs 119 bias the insert advancer 108 and the advancer release 105 to respective default positions, and actuation of the trigger 104 or the advancer release 105 counteracts the bias imparted by the spring 119, and release of the trigger 104 or the advancer release 105 allows the spring 119 to return the trigger 104 or the advancer release 105 to the default position according to the bias of the spring 119.

Although the handle 110 in FIGS. 1A and 1B is shown with a “pistol grip” form factor, other embodiments may include other form factors for the handle 110, such as modified pistol grips (e.g., where the handle is oriented at a different angle relative to the longitudinal axis (in the Y direction as illustrated) of the needle 114 than that illustrated), T-grips (e.g., with two or more pistol style or modified pistol style grips at different arc positions around the longitudinal axis of the needle 114), U-grips (e.g., T-grips in which at least two adjacent extensions from the longitudinal axis of the needle 114 are linked via an intervening member), mug grips (e.g., two pistol grips or modified pistol grips defined at different distances along the longitudinal axis of the needle 114 and linked via an intermediate member), or the like. Although not illustrated, various ergonomic and friction increasing elements may be included on the handle 110, such as finger detents, rubberized portions, hatching, and the like.

In various embodiments, a syringe 109 is connected on a second end of the aspiration insert 102 (with the first end, opposite to the second end, being inserted in the needle 114). The syringe 109 includes a lumen in which a plunger 111 is disposed, that an operator can draw outward from the lumen to exert negative pressure (e.g., suction) via the aspiration insert 102 on a BMA harvesting site. As a benefit of the illustrated design, an operator can hold the handle 110 (and actuate the trigger 104) with one hand while the other hand is free to draw the plunger 111 from the syringe 109, thereby simplifying the harvesting technique compared to prior art BMA harvesters, and reducing the total number of harvesting steps.

FIGS. 2A-2C illustrate detailed views of the needles 114 of a harvesting device 100, according to embodiments of the present disclosure. The tip of the needle 114 is closed and includes a space in the lumen that is forward of any of the lateral holes 101 to allow a portion of the aspiration insert 102 that is forward of any aspiration windows 103 to seat in the space and thereby aligns the aspiration windows 103 with the first lateral holes 101 defined in the second direction from the trocar 116, as is shown in FIG. 2A.

FIGS. 2A-2C also illustrates optional gaskets 210 (e.g., such as O-rings) placed before and after the aspiration windows 103. When the aspiration insert 102 is positioned within the needle 114 to align the aspiration window 103 with the lateral needle holes 101, the presence of gaskets 210 on the aspiration insert 102 creates a seal that only allows BMA aspiration from the aligned lateral holes 101. Additionally or alternatively, the outer diameter of the aspiration insert 102 is designed to provide enough clearance with the inner diameter of the needle 114 to allow movement, but small enough to restrict BMA flow without the use of gaskets.

During operation, the operator actuates the trigger 104 to pull the aspiration insert 102 a predefined distance outward from the needle 114. For example, when a first set of lateral holes 101 is located a certain distance from a second set of lateral holes (e.g., the adjacent lateral holes 101), the trigger 104 pulls the aspiration insert 102 back by that certain distance so that the aspiration windows 103 are repositioned from being aligned with the first lateral holes 101 (as in FIG. 2A) to being aligned with the second lateral holes 101 (as in FIG. 2B). Mechanical feedback features may be used to provide the operator with an audible or tactile signal that indicates the aspiration windows 103 are properly aligned with the design set of lateral holes 101. The operator may continue actuating the trigger 104, each time advancing the alignment of the aspiration windows 103 to the next set of lateral holes 101 until the last set of lateral holes 101 is reached. In some embodiments, such as FIG. 1B, an aspiration insert stop 115 is integrated in the handle 110. Once the aspiration insert collet 106 reaches the aspiration insert stop 115, the aspiration insert 102 can no longer move, leaving the aspiration window 103 aligned on the last lateral hole 101. In other embodiments, such as in FIG. 2C, the aspiration insert 102 can continue to be advanced beyond the last set of lateral holes 101 to align the aspiration windows with a sealed portion of the needle 114 (e.g., lacking lateral holes 101) to prevent over-aspirating a BMA harvesting site associated with the last set of lateral holes 101.

In various embodiments, the needles 114 are constructed with a trocar 116 at the tip. The trocar 116 may be integrated with the needle by machining the trocar feature and needle (with lateral holes and an internal lumen) from a single piece of material. Alternatively, the trocar tip 116 can be welded onto a needle tube, or the end of a needle tube can be heated and compressed to form a solid trocar tip. Similarly to the needle 114, the aspiration insert 102 is closed on the first end to prevent the intake of BMA (and other materials) from a previously harvested BMA site. In some embodiments, the needle 114 includes one or more gaskets 210 (e.g., O-rings) to seal the aspiration windows 103 from suctioning BMA from other lateral holes 101 besides those that the aspiration windows 103 are currently aligned with.

In various embodiments, as an alignment feature, the needle 114 includes an alignment space of a given length defined in the lumen between the trocar 116 and the first set of lateral holes 101 and the aspiration insert 102 includes a seal spacing tip of that given length between a first end of the aspiration insert 102 and the aspiration window 103. Accordingly, an operator may push the aspiration insert 102 into the lumen of the needle 114, and the aspiration insert 102 contacts the trocar 116 (and resists further insertion) when the seal spacing tip occupies the alignment space in the lumen of the needle 114. Accordingly, when the seal spacing tip occupies the alignment space in the lumen of the needle 114, the aspiration window(s) is/are aligned with the first set of lateral holes 101, thereby readying the surgical device for BMA harvesting and ensuring that when the operator actuates the trigger that the aspiration window(s) 103 advance to the next set of lateral holes 101 and will form seals with those lateral holes 101.

FIGS. 3A and 3B illustrate cross-sectional views of a needle 114 for a harvesting device 100 taken in different planes perpendicular to the longitudinal axis of the needle 114, according to embodiments of the present disclosure. FIG. 3A illustrates a longitudinal view of a needle 114 with a first cross-sectional plane 310a, a second cross-sectional plane 310b, and a third cross-sectional plane 310c shown. Each of the cross-sectional planes 310a-c are perpendicular to the plane in which the needle 114 is illustrated (e.g., in XY planes relative to a YZ plane).

FIG. 3B illustrates several cross-sectional views of lateral holes options illustrated in a first cross-sectional plane 310a, a second cross-sectional plane 310b, and a third cross-sectional plane 310c. A first set 320a shows the equidistant placement of two lateral holes 101 opposing one another and spaced 180 degrees apart. A second set 320b shows the equidistant placement of three lateral holes 101 spaced 120 degrees apart, with the set of three lateral holes 101 in plane B offset by a rotation of 60 degrees relative to the sets of three lateral holes 101 in plane A and plane C. A third set 320c shows the equidistant placement of four lateral holes opposing one another and spaced 90 degrees apart. Other sets with more or fewer lateral holes at different angles, rotations, and bore sizes may be provided in other embodiments.

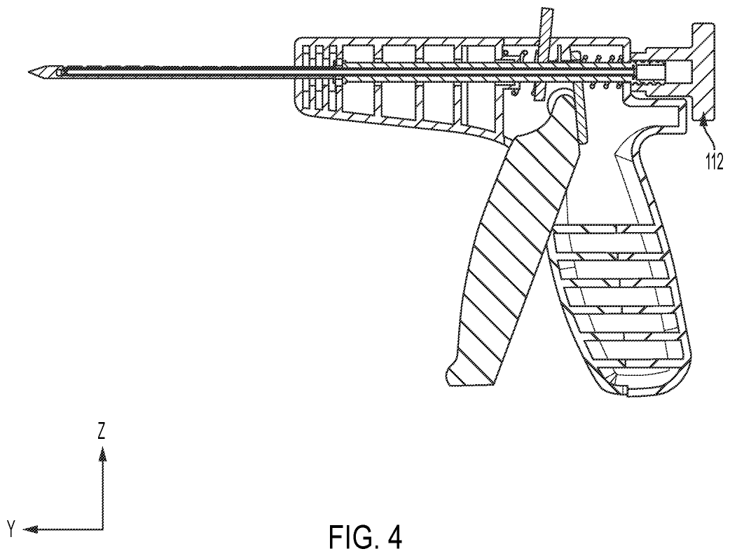

FIG. 4 illustrates an impact plate 112 for use in inserting a harvesting device 100 into an extraction site, according to embodiments of the present disclosure. In various embodiments, before the syringe 109 is attached to the aspiration insert 102 (or after the syringe 109 is detached to reposition where the needle 114 is inserted), an impact plate 112 is attached to the butt of the handle 110 in line with the needle 114.The impact plate 112 is configured to transfer percussive force applied to the impact plate 112 (e.g., via a surgical hammer) along the longitudinal axis of the needle 114 to the trocar tip 116 to drive the harvesting device 100 to a desired depth in a bone from which BMA is to be harvested. Once the harvesting device 100 reaches the desired depth, the operator may remove the impact plate 112 and attach the syringe 109 to the aspiration insert 102.

In various embodiments, the aspiration insert 102 may be held in the lumens of the aspiration insert collet 106 and the needle 114 while the impact plate 112 is installed, or may be omitted until the operator removes the impact plate 112. For example, although illustrated as a cap style impact plate 112 in FIG. 4, in some embodiments, the impact plate 112 may include a column adapted to be inserted into the harvesting device 100 where the aspiration insert 102 is located, to contact the distal end of the lumen of the needle 114 to transfer percussive force directly to the trocar 116.

In some embodiments, the impact plate is integrated into the handle design and is not removable. In such embodiments, the percussive force is applied to impact area designed into the handle.

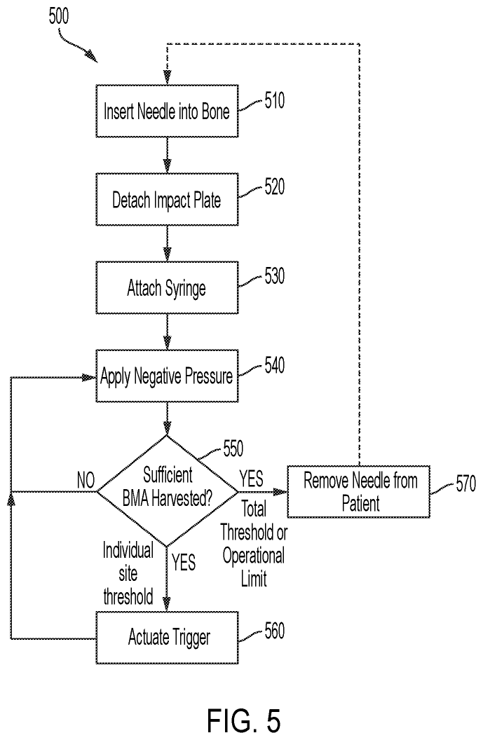

FIG. 5 is a flowchart of a method 500 for BMA harvesting using a described surgical device, according to embodiments of the present disclosure. Method 500 begins at operation 510, where an operator inserts the needle of the surgical device into a bone via percussive force applied to an impact plate connected to the surgical device. In various embodiments, the operator may select needles of different lengths, bore sizes, and number of lateral holes defined in the wall of the needle to define different harvesting sites for BMA from different cancellous bones (e.g., iliae, tibiae, vertebrae, calcanea, etc.). In various embodiments, the operator may apply the percussive force to the impact plate via a surgical hammer to drive the needle into the bone tissue of the patient.

At operation 520, the operator detaches and removes the impact plate from the surgical device once the needle is aligned to position the lateral holes with the bone marrow collection sites in the bone. Needle position may be visually determined using intraoperative radiographic imagining (e.g., fluoroscope imaging).

At operation 530, the operator attaches a syringe to a free end (e.g., the end not inserted in the needle) of the aspiration insert. In some embodiments, the operator ensures that the aspiration insert is fully seated in the needle, such that a space forward of the first set of lateral holes in the needle accepts a closed spacer of the aspiration insert that is forward of the aspiration window. In various embodiments, the operator may attach the syringe to an aspiration insert that was present in the surgical device while the impact plate was attached, or may introduce an aspiration tube to the surgical device after detaching and removing the impact plate.

At operation 540, the operator applies a negative pressure to the current bone marrow collection site via the aspiration insert. In some embodiments, the negative pressure is generated by the operator pulling a plunger on the syringe outward from a lumen of the syringe, although other negative pressure sources (e.g., vacuum pumps) and alternate collection containers can be used in other embodiments. The negative pressure exerts suction on the tissue surrounding the lateral holes aligned with having the aspiration windows to draw BMA into the syringe via the aspiration insert.

At operation 550, the operator determines whether sufficient BMA has been harvested from the current bone marrow collection location. In various embodiments, the operator may determine that sufficient BMA has been collected according to an individual location threshold amount (e.g., between 1-2 cc) of BMA has been harvested from the current site when the aspiration windows are aligned with a final set of lateral holes (e.g., further from the tip of the needle), or a predefined amount of BMA has been collected from across multiple bone marrow collection locations accessed by aspiration at multiple lateral holes on the needle. When the individual site threshold amount has not been reached, method 500 returns to operation 540 to continue applying negative pressure to the current collection site. When the individual site threshold amount has been reached, method 500 proceeds to operation 560 for the operator to select a new collection location accessed by a new set of lateral holes on the needle. When the aspiration windows are aligned with a final set of lateral holes (e.g., further from the tip of the needle), or a predefined amount of BMA has been collected from across multiple bone marrow collection sites, method 500 proceeds to operation 570 to remove the needle from the patient.

At operation 560, the operator actuates a trigger to pull the aspiration insert by a certain distance relative to the needle while the needle remains stationary relative to the current bone marrow collection site. Because the lateral holes of the needle are also located a certain distance along the wall of the needle from at least one adjacent lateral hole, actuating the trigger aligns the aspiration window with a second lateral hole defined in the needle to move the current harvesting site from a first site (closer to the tip of the needle) to a second site (further from the tip of the needle). Method 500 may then return to operation 540 to apply suction to the surrounding tissue at a new current bone marrow collection location accessed by the newly aligned lateral hole. Because the trigger is integrated in the handle that the operator uses to hold the surgical device in place, the operator may advance the harvesting site using one hand; leaving the operator's second hand free to concurrently apply the negative pressure to the collection site, such as when using an aspiration syringe.

At operation 570, the operator removes the needle from the patient. In various embodiments, the operator may fully remove the needle from the patient when the operator has collected a predefined amount of BMA from the patient. In some embodiments, when the operator has not collected the predefined amount of BMA from the patient, the operator may remove the syringe and reattach the impact plate, to either drive the needle into a new location in the same bone (e.g., at another entry point, at a different angle from the same entry point) or a new location in a different bone. Accordingly, method 500 may conclude after operation 570 or return to operation 510.

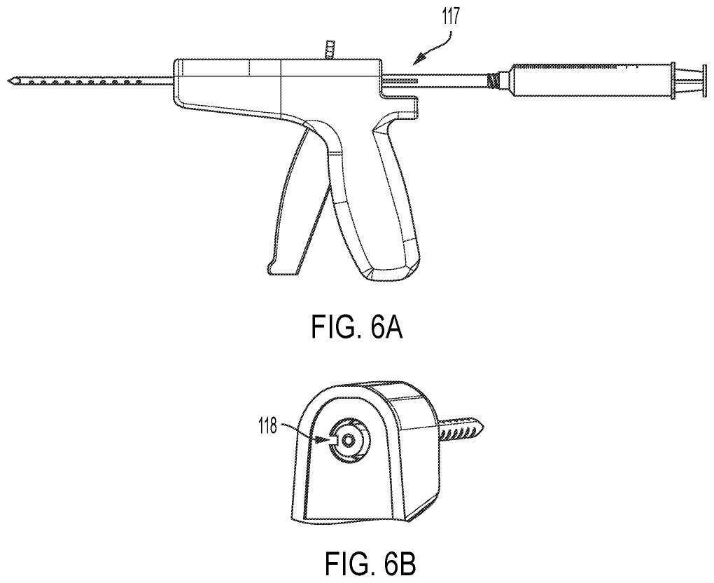

FIG. 6A and 6B illustrate an alignment groove 117 present on the aspiration insert collet 106 that mates with an alignment pin 118 found on a reinforcement strut 113. During assembly of the device 100, the aspiration insert collet 106 is placed in the handle 110 such that the alignment pin 118 seats within the alignment groove 117. The alignment pin 118 prevents the aspiration insert collet 106 and embedded aspiration insert 102 from rotating. This maintains the rotational alignment of the aspiration window 103 with the lateral holes 101 on the needle 114 each time the aspiration insert 102 is moved to a new position.

The present disclosure may also be understood with reference to the following numbered clauses.

Clause 1: A surgical device, comprising: a needle including a plurality of lateral holes defined at different positions longitudinally along a wall of the needle, wherein each lateral hole of the plurality of lateral holes is located a certain distance from at least one adjacent lateral hole of the plurality of lateral holes; an aspiration insert including an aspiration window that is sized to interface with one set of lateral holes of the plurality of lateral holes at a time, wherein the aspiration insert is disposed in a lumen of the needle; and a handle including a trigger in communication with the aspiration insert, wherein a positioning means (e.g., trigger, ratchet, knob, etc.) is configured to actuate between a first state and a second state to move the aspiration insert a certain distance in a first direction along a longitudinal axis of the needle.

Clause 2: The surgical device of any of clauses 1 and 3-12, further comprising: a syringe connected to a first end of the aspiration insert opposite to a second end of the aspiration insert inserted in the needle.

Clause 3: The surgical device of any of clauses 1, 2, and 4-12, further comprising: an impact plate configured to connect to the handle in line with the needle to transfer percussive force applied along the longitudinal axis to a trocar tip of the needle.

Clause 4: The surgical device of any of clauses 1-3 and 5-12 further comprising: an impact plate integrated with the handle to transfer percussive force applied along the longitudinal axis to a trocar tip of the needle.

Clause 5: The surgical device of any of clauses 1-4 and 6-12, wherein the aspiration window interfaces with one lateral hole at a time by forming a seal with the one lateral hole to define a fluid pathway from outside of the lumen of the needle into a lumen of the aspiration insert.

Clause 6: The surgical device of any of clauses 1-5 and 7-12, wherein the wall of the needle is at least 0.38 millimeters (mm) thick.

Clause 7: The surgical device of any of clauses 1-6 and 8-12, wherein the needle further includes an integrated trocar tip.

Clause 8: The surgical device of any of clauses 1-7 and 9-12, wherein the needle further includes a gasket around the aspiration window.

Clause 9: The surgical device of any of clauses 1-8 and 10-12, wherein the plurality of lateral holes defined at the different positions along the longitudinal axis of the needle is grouped with a second plurality of lateral holes defined at the different positions longitudinally along the needle at a rotational offset around a circumference of the needle relative to the plurality of lateral holes.

Clause 10: The surgical device of any of clauses 1-9, 11, and 12, wherein the needle further includes a space of a given length defined in the lumen between a trocar and a first set of lateral holes, wherein the aspiration insert includes a seal spacing tip of the given length between a first end and the aspiration window.

Clause 11: The surgical device of any of clauses 1-10 and 12, further comprising: a negative pressure source and collection container connected to the aspiration insert.

Clause 12: The surgical device of any of clauses 1-11, wherein the negative pressure source and collection device is a syringe.

Clause 13: A method, comprising: applying a negative pressure to a first bone marrow collection site via an aspiration insert having an aspiration window aligned with a first set of lateral holes defined in a needle in which the aspiration insert is disposed; actuating a positioning means to retract the aspiration insert by a certain distance relative to the needle while the needle remains stationary relative to the first bone marrow collection site to align the aspiration window with a second lateral hole defined in the needle; and applying the negative pressure to a second bone marrow collection site via the aspiration insert when aligned with the second lateral hole defined in the needle.

Clause 14: The method of any of clauses 13 and 15-20, further comprising, before applying the negative pressure to the first bone marrow collection site: inserting the needle into a bone via percussive force applied to an impact plate connected to a surgical device in which the needle is included; and detaching the impact plate from the surgical device once the needle is aligned to position the first lateral hole with the first bone marrow collection site and the second lateral hole with the second bone marrow collection site.

Clause 15: The method of any of clauses 13, 14, and 16-20, wherein the negative pressure is applied by a syringe in which bone marrow aspirate is collected via the aspiration insert from the first bone marrow collection site and the second bone marrow collection site.

Clause 16: The method of any of clauses 13-15 and 17-20, further comprising: in response to collecting a predefined amount of bone marrow aspirate from the first bone marrow collection site and the second bone marrow collection site, removing the needle from a bone that includes the first bone marrow collection site and the second bone marrow collection site.

Clause 17: The method of any of clauses 13-16 and 18-20, further comprising: actuating a positioning means to retract the aspiration insert by the certain distance relative to the needle while the needle remains stationary relative to the first bone marrow collection site to align the aspiration window with a third lateral hole defined in the needle; and applying the negative pressure to a third bone marrow collection site via the aspiration insert when aligned with the third lateral hole defined in the needle.

Clause 18: The method of any of clauses 13-17, 19, and 20, further comprising: while applying the negative pressure and not actuating the trigger, holding the aspiration insert in place relative to the needle via an advancer release and an insert advancer.

Clause 19: The method of any of clauses 13-18 and 20, wherein the needle includes a third lateral hole and a fourth lateral hole and the aspiration insert includes a second aspiration window, wherein the second aspiration window is aligned with the third lateral hole when the aspiration window is aligned with the first lateral hole and the second aspiration window is aligned with the fourth lateral hole when the aspiration window is aligned with the second lateral hole.

Clause 20: The method of any of clauses 13-19, further comprising: attaching a negative pressure source and collection container to a first end of the aspiration insert, wherein a second end of the aspiration insert is disposed in the needle.

Clause 21: A surgical device, comprising: a needle including a first lumen and a plurality of sets of lateral holes defined at a plurality of positions in a wall of the needle, wherein each lateral hole of a given set of lateral holes defined at a given position of the plurality of positions is defined circumferentially around the wall, and wherein each position of the plurality of positions is located a certain distance longitudinally along the needle from at least one adjacent position of the plurality of positions; an aspiration insert, disposed in the first lumen, and including a second lumen and an aspiration window; and a handle including a positioning means configured to, upon actuation, reposition the aspiration window from alignment with a first set of lateral holes of the plurality of sets of lateral holes defined at a first position of the plurality of positions to a second set of lateral holes of the plurality of sets of lateral holes to place the second lumen in fluid communication with a different portion of an environment outside of the first lumen.

Clause 22: The surgical device of any of claims 21, 23, and 24, wherein a first end of the aspiration insert is disposed within the needle and a second end of the aspiration insert, opposite to the first end, is configured to attach to a syringe.

Clause 23: The surgical device of any of claims 21, 22, and 24, wherein a negative pressure source and collection container is connected to the aspiration insert.

Clause 24: The surgical device of any of claims 21, 22, and 23, wherein the negative pressure source and collection device is a syringe.

The descriptions and illustrations of one or more embodiments provided in this disclosure are intended to provide a thorough and complete disclosure the full scope of the subject matter to those of ordinary skill in the relevant art and are not intended to limit or restrict the scope of the subject matter as claimed in any way. The aspects, examples, and details provided in this disclosure are considered sufficient to convey possession and enable those of ordinary skill in the relevant art to practice the best mode of the claimed subject matter. Descriptions of structures, resources, operations, and acts considered well-known to those of ordinary skill in the relevant art may be brief or omitted to avoid obscuring lesser known or unique aspects of the subject matter of this disclosure. The claimed subject matter should not be construed as being limited to any embodiment, aspect, example, or detail provided in this disclosure unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently.

Having been provided with the description and illustration of the present disclosure, one of ordinary skill in the relevant art may envision variations, modifications, and alternate embodiments falling within the spirit of the broader aspects of the general inventive concept provided in this disclosure that do not depart from the broader scope of the present disclosure.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

As used in the present disclosure, the terms “substantially”, “approximately”, “about”, and other relative terms encompass values within ±5% of a stated quantity, percentage, or range unless a different approximation is explicitly recited in relation to the state quantity, percentage, or range or if the context of the value indicates that a different approximation would be more appropriate. For example, a value identified as about X % may be understood to include values between 0.95*X % and 1.05*X % or between X-0.05X and X+0.05X percent, but may stop at zero or one hundred percent in various contexts. In another example, a feature described as being substantially parallel or perpendicular to another feature shall be understood to be within ±9 degrees of parallel or perpendicular. Any value stated in relative terms shall be understood to include the stated value and any range or subrange between the indicated or implicit extremes.

As used in the present disclosure, all numbers given in the examples (whether indicated as approximate or otherwise) inherently include values within the range of precision and rounding error for that number. For example, the number 4.5 shall be understood to include values from 4.45 to 4.54, while the number 4.50 shall be understood to include values from 4.495 to 4.504. Additionally, any number or range that explicitly or by context refers to an integer amount (e.g., approximately X users, between about Y and Z states), shall be understood to round downward or upward to the next integer value (e.g., X±1 users, Y−1 and Z+1 states).

The following claims are not intended to be limited to the embodiments shown herein, but are to be accorded the full scope consistent with the language of the claims. Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various aspects described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

We claim:

1. A surgical device, comprising:
a needle including a plurality of lateral holes defined at different positions longitudinally along a wall of the needle, wherein each lateral hole of the plurality of lateral holes is located a certain distance from at least one adjacent lateral hole of the plurality of lateral holes;
an aspiration insert including an aspiration window that is sized to interface with one lateral hole of the plurality of lateral holes at a time, wherein the aspiration insert is disposed in a lumen of the needle; and
a handle including positioning means in communication with the aspiration insert, wherein the positioning means is configured to actuate between a first state and a second state to move the aspiration insert a certain distance in a first direction along a longitudinal axis of the needle.

2. The surgical device of claim 1, further comprising:
a syringe connected to a first end of the aspiration insert opposite to a second end of the aspiration insert inserted in the needle.

3. The surgical device of claim 1, further comprising:
an impact plate configured to connect to the handle in line with the needle to transfer percussive force applied along the longitudinal axis to a trocar tip of the needle.

4. The surgical device of claim 1, further comprising:
an impact plate integrated in the handle to transfer percussive force applied along the longitudinal axis to a trocar tip of the needle.

5. The surgical device of claim 1, wherein the aspiration window interfaces with one lateral hole at a time by forming a seal with the one lateral hole to define a fluid pathway from outside of the lumen of the needle into a lumen of the aspiration insert.

6. The surgical device of claim 1, wherein the wall of the needle is at least 0.38 millimeters (mm) thick.

7. The surgical device of claim 1, wherein the needle further includes an integrated trocar tip.

8. The surgical device of claim 1, wherein the needle further includes a gasket around the aspiration window.

9. The surgical device of claim 1, wherein the plurality of lateral holes defined at the different positions along the longitudinal axis of the needle is grouped with a second plurality of lateral holes defined at the different positions longitudinally along the needle at a rotational offset around a circumference of the needle relative to the plurality of lateral holes.

10. The surgical device of claim 1, wherein the needle further includes a space of a given length defined in the lumen between a trocar and a first set of lateral holes, wherein the aspiration insert includes a seal spacing tip of the given length between a first end and the aspiration window.

11. The surgical device of claim 1, further comprising:
a negative pressure source and collection container connected to the aspiration insert.

12. The surgical device of claim 11, wherein the negative pressure source and collection device is a syringe.

13. A method, comprising:
applying a negative pressure to a first bone marrow collection site via an aspiration insert having an aspiration window aligned with a first lateral hole defined in a needle in which the aspiration insert is disposed;
actuating a positioning device to retract the aspiration insert by a certain distance relative to the needle while the needle remains stationary relative to the first bone marrow collection site to align the aspiration window with a second lateral hole defined in the needle; and
applying the negative pressure to a second bone marrow collection site via the aspiration insert when aligned with the second lateral hole defined in the needle.

14. The method of claim 13, further comprising, before applying the negative pressure to the first bone marrow collection site:
inserting the needle into a bone via percussive force applied to an impact plate connected to a surgical device in which the needle is included; and
detaching the impact plate from the surgical device once the needle is aligned to position the first lateral hole with the first bone marrow collection site and the second lateral hole with the second bone marrow collection site.

15. The method of claim 13, wherein the negative pressure is applied by a syringe in which bone marrow aspirate is collected via the aspiration insert from the first bone marrow collection site and the second bone marrow collection site.

16. The method of claim 13, further comprising:
in response to collecting a predefined amount of bone marrow aspirate from the first bone marrow collection site and the second bone marrow collection site, removing the needle from a bone that includes the first bone marrow collection site and the second bone marrow collection site.

17. The method of claim 13, further comprising:
actuating the positioning device to retract the aspiration insert by the certain distance relative to the needle while the needle remains stationary relative to the first bone marrow collection site to align the aspiration window with a third lateral hole defined in the needle; and
applying the negative pressure to a third bone marrow collection site via the aspiration insert when aligned with the third lateral hole defined in the needle.

18. The method of claim 13, further comprising:
while applying the negative pressure and not actuating the positioning device, holding the aspiration insert in place relative to the needle via an advancer release and an insert advancer.

19. The method of claim 13, wherein the needle includes a third lateral hole and a fourth lateral hole and the aspiration insert includes a second aspiration window, wherein the second aspiration window is aligned with the third lateral hole when the aspiration window is aligned with the first lateral hole and the second aspiration window is aligned with the fourth lateral hole when the aspiration window is aligned with the second lateral hole.

20. The method of claim 13, further comprising:
attaching a negative pressure source and collection container to a first end of the aspiration insert, wherein a second end of the aspiration insert is disposed in the needle.
